(19) United States
(12) Patent Application Publication
(10) Pub. No.: US 2013/0203953 A1
(43) Pub. Date: Aug. 8, 2013

(54) METHOD FOR THE PRODUCTION OF OLEFINS, AN OLEFIN, A POLYOLEFIN, AND USE OF THE POLYOLEFIN

(71) Applicants: Gonçalo Amarante Guimarães Pereira, Campinas (BR); Johana Rincones Perez, Campinas (BR); Marcelo Falsarella Carazzolle, Valinhos (BR); Antonio Luiz Ribeiro de Castro Morschbacker, Campinas (BR); Luiza Roza, Porto Alegre (BR); Márcio Henrique dos Santos Andrade, Maceio (BR)

(72) Inventors: Gonçalo Amarante Guimarães Pereira, Campinas (BR); Johana Rincones Perez, Campinas (BR); Marcelo Falsarella Carazzolle, Valinhos (BR); Antonio Luiz Ribeiro de Castro Morschbacker, Campinas (BR); Luiza Roza, Porto Alegre (BR); Márcio Henrique dos Santos Andrade, Maceio (BR)

(21) Appl. No.: 13/513,764

(22) PCT Filed: Dec. 3, 2010
(86) PCT No.: PCT/BR10/00401

§ 371 Date: Apr. 15, 2013

(30) Foreign Application Priority Data
Dec. 4, 2009 (BR) PI0904979-7

(51) Int. Cl. C12P 5/02

(52) U.S. Cl. CPC C12P 5/02
USPC 526/351; 435/167; 526/352; 585/16

(57) ABSTRACT

The present invention relates to a method for the production of an olefin from at least one renewable natural raw material. More specifically, the present invention refers to a method whereby is obtained ethylene or propylene at high yield and high productivity by means of the anodic electrodecarboxylation reaction of carboxylic acids, respectively propionic acid and butyric acid, produced from fermentation, preferably of sugars. The method for generating the olefin is simple, has a low cost, and provides low emissions of greenhouse gasses of fossil origin.

### FIELD OF THE INVENTION

The present invention relates to a method for the production of an olefin from at least one natural renewable raw material. More specifically, the present invention refers to a process whereby ethylene or propylene is obtained with high yield and high productivity by means of an anodic electro-decarboxylation reaction of carboxylic acids, respectively propionic acid or butyric acid, produced by fermentation, preferably of sugars. The process for generating the olefin is simple, low-cost, and entails a low level of emission of greenhouse gases of fossil origin.

### BACKGROUND OF THE INVENTION

Ethylene and propylene are olefins that are mainly produced as byproducts of petroleum refining, by means of vapor reformation or catalytic cracking. (Ethylene, Michael T. Devanney, CEH Marketing Research Report—2005; Propylene, Jamie G. Lacson, CEH Marketing Research Report—2004).

Ethylene and propylene may be used as raw materials for the production of a wide range of products. For example, ethylene and propylene may be polymerized to form thermoplastic resins, respectively polyethylene and polypropylene. Such resins have applications in rigid and flexible packages, in the conformation of pieces obtained by blow molding, injection molding and rotational molding [rotomolding]. Additionally, they may be used as coating for carton packages and in the provision of adhesive layers. The various types of polyethylenes and polypropylenes represent, as a whole, the segment of most intensively produced and used thermoplastic resins in the world.

The global interest for organic products produced from renewable sources has increased greatly over the last few years, particularly in the case of plastics. Biopolymers that may be obtained from sugar sources, such as poly(lactic acid) and polyhydroxy butyrate, constitute some of the few available alternatives. More recently, green polyethylene produced from ethylene monomers derived from a natural renewable raw material source, such as sugar cane ethanol, has become available with great success. The incineration of this type of product does not cause fossil carbon dioxide emissions.

The use of products obtained from natural raw materials, rather than those obtained from fossil sources has become an increasingly acceptable proposal for reducing and mitigating fossil greenhouse gas emissions. These products effectively combat growing intensity of the greenhouse effect. The products obtained from natural materials may be certified as to their renewable carbon content in accordance with the methodology described in the technical standard ASTM D 6866-06, “Standard Test Methods for Determining the Biobased Content of Natural Range Materials Using Radiocarbon and Isotope Ratio Mass Spectrometry Analysis”.

Another advantage derived from the use of renewable raw materials in the production of thermoplastic resins is the fact that such practice decreases the use of the increasingly scarce fossil carbon sources. In the case of polymers, particularly of polyolefins, their numerous applications evidence the great potential of these products for carbon dioxide mitigation if obtained from renewable raw materials. In addition to saving fossil resources and their capacity to capture carbon dioxide from the atmosphere, the decomposition of green polyolefins by incineration does not release fossil carbon into the atmosphere.

Propionic acid is commercially produced from raw petrochemical source materials. One method of obtaining propionic acid is from petrochemical ethylene, carbon monoxide and steam. This organic acid may also be produced by oxidation of propionaldehyde obtained by the oxo synthesis, also starting from ethylene. Propionic acid may also be produced from natural carbon sources, such as sugars, glycerol and starch, in the presence of bacteria of certain genera, such as those of the genus Propionibacterium, for example the species Propionibacterium acidipropionici and Propionibacterium freudenreichii. (Boyaval P., Cone C. Production of propionic acid, Lait (1995) 75, 453-461, Elsevier.). However, this fermentation is only commercially exploited for the production of certain types of cheese, since the low concentration of the fermentation product, its low productivity (<1 g/L.h) and the difficulty to remove propionic acid from fermentation broth render this route not very competitive from the commercial stand point.

Butyric acid is commercially produced from petrochemical propylene. Firstly, butyraldehyde is produced by the oxo synthesis of propylene, followed by oxidation to butyric acid. Butyric acid may also be produced from natural carbon sources, such as sugars, glycerol and starch, in the presence of bacteria of certain genera, such as those of the genus Clostridium, for example, the species Clostridium tyrobutyricum (Zigová, J., Sturdik, E., Advances in biotechnological production of butyric acid, Journal of Industrial Microbiology & Biotechnology (2000) 24, 153-160), Clostridium butyricum, Clostridium acetobutylicum and Clostridium thermobutyricum. Butyric acid is also found in certain milk byproducts, such as butter. This fermentation is not commercially exploited because the presence of byproducts, such as acetone and ethanol, the low concentration of the fermentation product and the difficulty to remove butyric acid from the fermentation broth render this route not very competitive from the commercial stand point.

In addition to the difficulties inherent to these two fermentation processes, the concentration and purification of the organic acids from the fermentation broth also represents a high-cost operation, due to the drop in pH and the low concentration of these products in the broth. Another aggravating circumstance to the processes of separation and purification is the fact that propionic and butyric acids have boiling points higher than water, which constitutes more than 90% of the medium. A series of works have been published aimed at facilitating and lowering the cost of removing the acids from the fermentation broth by way of various techniques. These studies aim at reducing the toxicity of these acids to the microorganisms and the inhibitory effect to fermentation caused by the decrease in pH. The low pH caused by the formation of the acids is often eliminated by the neutralization of the medium by the addition of a base, which however renders the process of recovery of the product both difficult and costly. In order to overcome these problems, several processes have been proposed, but they are complex, costly and fraught with operational difficulties in large scale and long terms, based, for example, in the use of membranes, precipitation with calcium salts or the extraction of the product from the medium with the use of solvents.

The anodic electro-decarboxylation of organic acids is a reaction known since the end of the XIXth century. Initially, the proposed use for this reaction was in the transformation of fatty acids into saturated hydrocarbons to be used as fuels.

For example, document U.S. Pat. No. 3,992,268 describes a process for the production of hydrocarbons from the fermentation of several types of residues resulting in a mixture of organic acids that are then converted, by way of the reaction known as Kolbe electrolysis, into a mixture of hydrocarbons and carbon dioxide.

The invention described in patent application No. WO 2007/095215 consists in using the reaction known as Kolbe electrolysis to produce mainly hexane and carbon dioxide and small amounts of propane and hydrogen from butyric acid obtained by fermentation. In this proposal, if the electrolysis was conducted in the untreated fermentation medium, the hexane thus obtained would be toxic to the microorganisms employed in the production of butyric acid, even though this compound exhibits low solubility in the aqueous medium. Moreover, this invention exhibits the additional problem of obtaining a liquid product that requires an additional operation for the removal of the product from the medium, thereby increasing the cost of the process.

In addition to the examples cited above, older records of the Kolbe electrolysis show that other byproducts are formed, such as ethane from acetic acid, decane from caproic acid and tetradecane from caprylic acid (Lob, W. Electrochemistry of Organic Compounds, Chapman and Hall, 1906).

Patent document No. WO 2008/103480 proposes the thermal decarboxylation, under supercritical vapor conditions, of polyhydroxy butyrate and/or carboxylic acids, such as butyric acid, for the production of hydrocarbons, mostly alkanes. The temperatures employed in the process are high, above 300 deg. C. or 400 deg. C. and the pressures employed are above atmospheric pressure.

Therefore, none of the references propose the use of a process of fermentation of sugars to organic acids, integrated with both the conversion of these acids to olefins and the removal of the obtained products from the medium in gaseous form. Similarly, none of the known references known to date proposes the use of a biological route for the production of carboxylic acids, wherein the control of the pH in the medium takes place by means of the production of olefins generated by the anodic decarboxylation of the organic acids thereof, and, if necessary, requiring a complementary addition of a base. The present invention has the purpose of producing olefins from renewable substrates, preferably from sugars, with high yield and high productivity, and enabling the easy removal of the olefins from the medium, in gaseous phase, without compromising the stability of the fermentation.

Therefore, it is possible to obtain olefins from renewable sources, with a low production cost and in which its life cycle, from planting of the natural renewable raw material until the production of the olefin, promotes the absorption of carbon dioxide from the atmosphere. The products obtained from these olefins, such as the polyethylene and the polypropylene, are capable of mitigating atmospheric carbon dioxide and, if incinerated, will generate non-fossil carbon dioxide.

### SUMMARY OF THE INVENTION

In light of what has been set forth, an embodiment of the present invention consists in the provision of a process for the production of ethylene or propylene from at least one renewable natural raw material.

Another embodiment of the present invention consists in the provision of a process for the production of ethylene or propylene from renewable carbon sources that is simple, has a low cost and entails a low level of emission of greenhouse gases of fossil origin.

Another embodiment of the present invention consists in the use of ethylene or propylene 100% originated from natural renewable carbon sources for the production of polymers and copolymers of ethylene and/or propylene. As a consequence of this fact and of the integration thereof with byproducts of the processing of the renewable natural raw material, and additionally, with byproducts of the present process, the olefins obtained according to the present invention promote, starting from the planting of the raw material, the mitigation of carbon dioxide from the atmosphere. The polymers obtained from these olefins, such as polyethylene and polypropylene, as well as the products manufactured from such polymers, generate carbon dioxide of non-fossil origin when incinerated.

Another embodiment of the present invention consists in the provision of an integrated process of fermentation of sugars to organic acids with the conversion of these acids to olefins and the consequent removal of the obtained products from the medium in gaseous form.

Another embodiment of the present invention consists in the provision of a biological route for the production of carboxylic acids, wherein the control of the pH in the medium takes place by means of the production of olefins generated by the anodic decarboxylation of the organic acids thereof, and, if necessary, requiring a complementary addition of a base, with the purpose of producing olefins from renewable substrates, preferably from sugars, with high yield and high productivity, and thus enabling the easy removal of the olefins from the medium, in gaseous form.

The present invention discloses a process for the production of an olefin from at least one renewable natural raw material. More specifically, the present invention relates to a process whereby ethylene or propylene is obtained from sugars, with high yield and high productivity, by means of an anodic decarboxylation reaction of carboxylic acids, propionic or butyric acids, respectively, produced from fermentation of at least one organic substrate of renewable natural origin, preferably from sugars.

The olefins produced according to the present invention are light olefins selected from the group that consists of ethylene and propylene. However, other olefins such as butylene or pentene can be obtained by way of the process according to the present invention, from valeric or caproic acids, respectively.

The processes for the production of ethylene or propylene according to the present invention are simple, have a low cost and may be advantageously employed in locations abundant with natural renewable raw materials, such as:
starch, such as corn;
cellulose and hemicellulose contained in lignocellulosic materials, such as leaves and bagasse;
impure glycerol, such as in the residue from saponification or biodiesel production processes;
residues containing lactose and/or lactates, such as whey;

and, preferably,
sugars, such as sugarcane.

Optionally, other natural renewable raw materials may be used as sugar sources, such as beetroot, and as starch sources, such as cassava, among others.

Propionic acid may be produced based on the use as substrate of various natural carbon sources, such as monosaccharides (both hexoses like glucose or fructose and pentoses like xylose), disaccharides (such as sucrose and lactose), hydrolysed polysaccharides (such as cellulose, hemicellulose and starch), glycerol or lactates in the presence of certain types of bacteria such as those of the genus Propionibacterium, such as, for example, the species Propionibacterium acidipropionici and Propionibacterium freudenreichii.

The propionic acid thus obtained is converted in the present invention to ethylene, by means of the anodic decarboxylation reaction, under conditions that maximize the formation of this olefin rather than producing other possible products of this reaction, such as, for example, n-butane.

In a similar manner, butyric acid may be produced based on the use as substrate of various natural carbon sources, such as sugars and starch, in the presence of certain types of bacteria such as those of the genus Clostridium such as, for example, the species Clostridium tyrobutyricum, Clostridium butyricum, Clostridium thermobutyricum and Clostridium acetobutylicum, and also directly from cellulose in the presence of other species of bacteria, such as, for example, the species Clostridium thermocellum and C. cellulolyticum.

The butyric acid thus produced is converted in the present invention to propylene by means of the anodic decarboxylation reaction under conditions that maximize the formation of this olefin rather than the production of other possible products of this reaction, such as, for example, hexane.

### BRIEF DESCRIPTION OF THE DRAWINGS

FIG. 1 represents a modality of the integrated process according to the present invention, whereby the sugars contained in the sugar cane juice are passed through a reactor wherein both the fermentation and the anodic electrodecarboxylation occur in the same section. In the fermentation, propionic acid is mainly produced, while carbon dioxide succinic acid and acetic acid are obtained as byproducts. As the organic acids accumulate in the fermentation broth, they are converted by means of the anodic electrodecarboxylation reaction, under conditions that promote the formation of ethylene, in addition to carbon dioxide and hydrogen. Small amounts of methane, ethane, butane and ethanol are also obtained. Part of the fermentation broth may be optionally cooled down in order to be thereafter recirculated and used in the dilution of further additions of sugar cane juice.

FIG. 2 represents another modality of the integrated process according to the present invention, whereby the sugars contained in the sugar cane juice are passed through a reactor comprising a lower fermentation section intended for the main production of propionic acid with carbon dioxide and a small amount of acetic and succinic acids as byproducts. The upper part of the reactor contains a section specifically intended for electrolysis wherein the electrodes perform the conversion of propionic acid under conditions that promote the formation of ethylene, with carbon dioxide and hydrogen as byproducts. All such formed products are volatile, and are therefore instantaneously withdrawn from the medium, thus keeping the pH within the desired range. Small amounts of methane, ethane, butane and ethanol are also obtained. A part of the fermentation broth is optionally cooled to be subsequently recirculated and used for the dilution of further additions of sugar cane juice.

FIG. 3 represents another modality of the integrated process according to the present invention, whereby the sugars contained in the sugar cane juice are firstly subjected to fermentation in a bioreactor in order to produce mainly butyric acid, with carbon dioxide (CO₂) and hydrogen (H₂) as the main byproducts. The fermentation broth containing butyric acid as its main product is then transferred from the bioreactor to an electrolyzation reactor in which the anodic decarboxylation reaction occurs, under conditions that promote the formation of propylene, in addition to carbon dioxide and hydrogen. Small amounts of propane, cyclopropane, hexane and propanol may also be obtained. The fermentation may also generate other byproducts, such as acetic acid, that may be converted to small amounts of minority products such as methane and ethane during the anodic decarboxylation reaction. A part of the fermentation broth is optionally cooled to be subsequently recirculated for the dilution of further additions of sugar cane juice.

FIG. 4 shows the gas chromatograms detected by (a) FID and (b) TCD after 6 hours of the experiment in example 1. Only the expected peaks (ethylene, H₂ and CO₂ from the anodic decarboxylation reaction, and N₂, from purge) were detected in all chromatograms obtained during the experiment.

FIG. 5 shows the HPLC chromatogram of the fermentation broth before (solid line) and after the anodic electro-decarboxylation reaction (dotted line) of the experiment in example 1. The absence of new peaks in the liquid phase after the reaction demonstrates that the consumed propionic acid was totally converted to compounds in gaseous form.

FIG. 6 shows the gas chromatograms detected by (a) FID and (b) TCD after 6 hours of the experiment in example 2. Only the expected peaks (propylene, H₂ and CO₂ from the anodic decarboxylation reaction, and N₂, from purge) were detected in all chromatograms obtained during the experiment.

FIG. 7 shows the HPLC chromatogram of the fermentation broth before (solid line) and after the anodic electro-decarboxylation reaction (dotted line) of the experiment in example 2. One new peak was observed in the liquid phase after the reaction at a retention time of 39 min. and may correspond to a small proportion of propanol formed as byproduct of the electro-decarboxylation reaction of butyric acid under the conditions used.

FIG. 8 shows the gas chromatograms detected by (a) FID and (b) TCD after 6 hours of the experiment in example 3. Only the expected peaks (propylene, H₂ and CO₂ from the anodic decarboxylation reaction, and N₂, from purge) were detected in all chromatograms obtained during the experiment.

FIG. 9 shows the HPLC chromatogram of the fermentation broth before (solid line) and after the anodic electro-decarboxylation reaction (dotted line) of the experiment in example 3. One new peak was observed in the liquid phase after the reaction at a retention time of 39 min. and may correspond to a small proportion of propanol formed as byproduct of the electro-decarboxylation reaction of butyric acid under the conditions used.

### DETAILED DESCRIPTION OF THE INVENTION

The present invention discloses a method for the production of olefins from at least one natural renewable raw material. More specifically, the present invention relates to a process whereby ethylene or propylene is obtained with high yield and high productivity by means of the anodic decarboxylation of carboxylic acids, propionic acid or butyric acid, respectively, produced from fermentation, mainly of sugars or other organic substrates from renewable sources.

Throughout the present text, the term “sugars” to be used in the present processes should be understood to refer to the sugars in solution, originating, for example, from sugar cane juice (containing sucrose, glucose and fructose) or from hydrolysed polysaccharides, such as cellulose (containing mainly glucose) or hemicellulose (containing mainly xylose and glucose). In this context, other sugars that may be used are hydrolyzed starch (containing glucose), or milk whey (containing lactose). Eventually, other renewable raw materials may be used as substrates in the fermentations of the present invention, such as glycerol residues from biodiesel or saponification processes, sorbitol or lactate.

The processes for the production of ethylene or propylene according to the present invention are simple, have a low cost, and may be advantageously employed in locations with ample availability of renewable natural raw materials rich in sugars, such as sugar cane; rich in starch, such as corn; rich in cellulose and hemicellulose, such as lignocellulosic materials; or consist of low cost raw materials, such as the glycerol found in biodiesel and saponification residues; or lactose and lactate found in whey residues from the processing of milk and dairy. Preferentially, the renewable natural raw materials employed are those that are rich in sugars and exhibit high biomass availability for co-generation of energy, such as sugar cane. Optionally, other renewable natural raw materials may be used as sugar sources, such as beetroot, and as starch sources, such as cassava, among others.

Eventually, by means of a process similar to that of the present invention, butylene and pentene maybe produced from valeric acid and caproic acid, respectively. These olefins may be used as co-monomers in the production of copolymers of ethylene and of propylene.

Propionic acid may be produced from natural carbon sources, such as sugars, glycerol, lactates or starch, in the presence of certain types of bacteria such as those of the genus Propionibacterium. In a preferred embodiment of the invention, the renewable natural raw material is constituted by materials rich in sugars, such as sugarcane, and the bacterium Propionibacterium acidipropionici is used in the fermentation, producing propionic acid as the main product, and carbon dioxide, acetic acid and succinic acid as the main byproducts. In another preferred embodiment of the invention, the renewable natural raw material is constituted by materials rich in sugars, such as hydrolysed lignocellulosic materials rich in glucose and xylose, with xylose being effectively transformed into propionic acid with high yield by the bacterium Propionibacterium acidipropionici, producing propionic acid as the main product, and carbon dioxide, acetic acid and succinic acid as the main byproducts (Ramsay J. A., Aly Hassan M. C., Ramsay B. A. Biological conversion of hemicellulose to propionic acid, Enzyme and Microbial Technology (1998) 22, 292-295, Elsevier). There are various known species of bacteria that are capable of metabolizing sugars and other substrates and convert them to propionic acid, and any of those may be used in the process according to the present invention, such as Propionibacterium thoenii, Propionibacterium freudenreichii, Propionibacterium jensenii, Propionibacterium acidifaciens, Propionibacterium shermanii, Selenomonas ruminantium, Propionigenium spp., Propionispira arboris, Clostridium propionicum, Megasphaera elsdenii and Bacteroies ruminicola. The fermentation typically takes place under anaerobic or microaerobic conditions.

Any type of fermenter may be used in the process according to the present invention. Bioreactors or fermenters with immobilized cells are preferentially used, since they present the advantage of maintaining a high concentration of cells in the medium and thus increase the productivity and yield of the fermentation. The immobilization of the microbial cells should be conducted with the use of materials that are inert to the fermentation system, such as calcium alginate, polyacrylamide gel and clays or through the use of membrane-type bioreactors.

Genetically modified microorganisms may be preferentially used for the purpose of reducing the formation of organic byproducts. One example of a genetically modified microorganism that is appropriate for the process according to the present invention is Propionibacterium acidipropionici ATCC 4875, a bacterium that produces propionic acid, acetic acid, succinic acid and carbon dioxide. In this bacterium, the genes of the metabolic route for the formation of acetic acid were eliminated to maximize the production and the yield of propionic acid (Zhang, A., et al., Biotechnology end Bioengineering, Wiley Periodicals, 2009).

Some of the important parameters in the fermentation according to the present invention are pH and temperature, and the optimal conditions for the fermentation of propionic acid are temperatures between 28 deg. C. and 45 deg. C., preferably between 30 deg. C. and 37 deg. C., and pH between 4.0 and 7.5, preferably between 5.0 and 7.0.

As far as it is know, a significant reduction of the pH value of the fermentation medium caused by the formation of the organic acids produces an inhibitory effect. Therefore, the fermentation of organic acids are normally conducted with rigid control of the pH value by means of the addition of bases, such as ammonium hydroxide, sodium hydroxide or calcium hydroxide, with the consequent formation of ammonium, sodium or calcium salts. In an embodiment of the present invention, the process may dispense the addition of bases for the control of the pH value of the medium, since the fermentation reaction is conducted with immediate removal of the carboxylic acids obtained from the fermentation medium by means of the anodic decarboxylation reaction, and thus eliminate the additional steps that are usually required for the recovery of the free organic acid. Optionally, and depending on the electro-decarboxylation conditions, a neutralizing base may also be added in order to maintain an optimal pH range. In this case, the additional steps required for the recovery of the organic acid are also eliminated, since the salt is also electro-decarboxylated to form the desired olefin.

A culture medium containing mineral salts and other nutrients is added to the fermentation system, and the stirring is maintained constant. The mineral salts and nutrients that are preferably used are, for example, sulfates, phosphates, nitrates, urea and ammonium, since these components do not influence the electrochemical step. Chlorides are to be avoided, since they interfere with the anodic decarboxylation reaction. When chlorides are necessary for the growth of the microorganism, an additional step for their removal may be added to the process, after the fermentation and before the electrochemical step. Chlorides may be removed by means of ion exchange resins, such as anionic or cationic resins or by processes such as electrodialysis or distillation.

In the case of bacteria of the genus Propionibacterium, their microaerobic nature dispenses a rigid control of anaerobic atmosphere of the bioreactor. The microaerobic or anaerobic condition of the system is maintained with a sprayer of nitrogen or, preferentially, of the carbon dioxide gas obtained as a byproduct of the fermentation and/or of the electrodecarboxylation reaction.

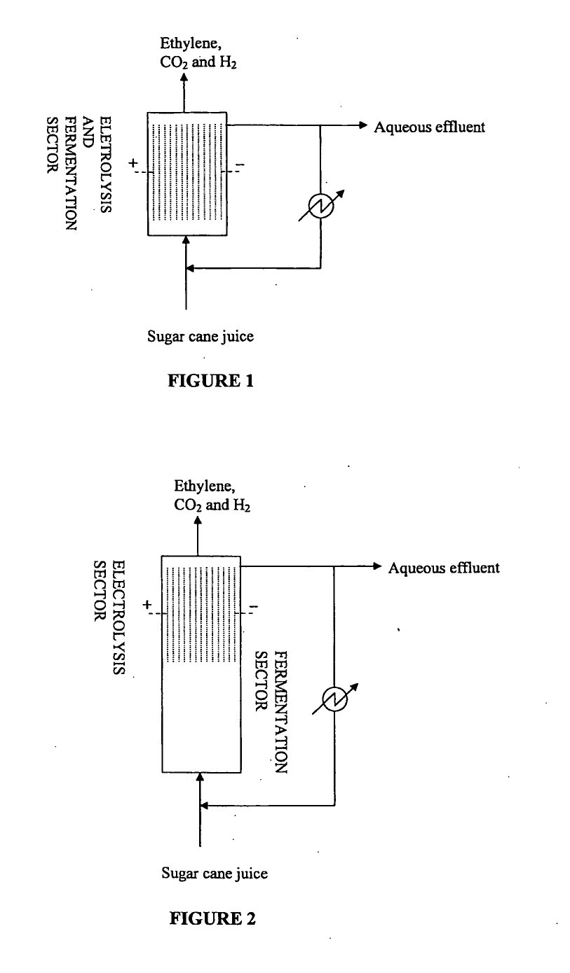

The anodic decarboxylation reaction of propionic acid may be conducted in the fermentation medium itself, where ethylene is produced and recovered, at high concentration, while the acid produced in the fermentation is consumed in the electrolysis. In an embodiment of the invention, the pH of the medium is naturally controlled by the continuous conversion of the carboxylic acid produced in the fermentation, with the consequent production of the ethylene by means of the anodic decarboxylation reaction and its continuous removal in gaseous form from the medium. Optionally, a neutralizing base may also be added in order to maintain an optimal pH range. When the anodic decarboxylation reaction occurs within the same vessel as the fermentation, it is pointed out that conditions such as pH, temperature, concentration of organic acids in the medium and composition of salts in the medium will be the same for the fermentation and at the beginning of the electrolysis. Optionally, a membrane or diaphragm type separator may be used for the purpose of separating the fermentation chamber from the electrolysis chamber. The anodic decarboxylation reaction in the fermentation medium itself may occur either in the same section wherein the fermentation occurs, as shown in FIG. 1, or in a section apart, located contiguous to the fermentation region, as shown in FIG. 2.

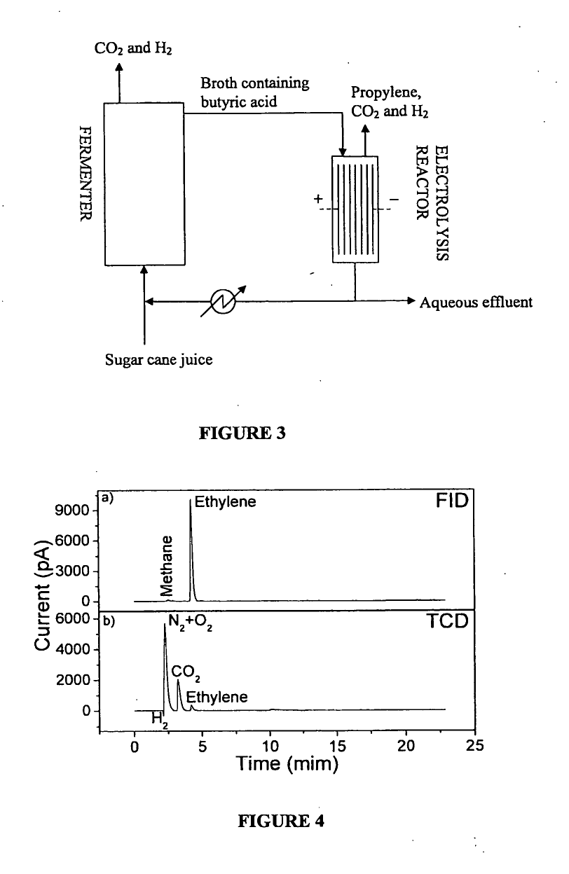

Alternatively, propionic acid may be electrodecarboxylated separately after conclusion of the fermentation, as shown in FIG. 3. In such circumstance, the fermentation broth is conveyed to an electrochemical cell located after the fermenter, wherein the production of the ethylene occurs, together with carbon dioxide (CO₂), hydrogen (H₂) and, optionally and preferentially, the subsequent recirculation into the fermenter of the reacted fermentation broth. In this case, it is pointed out that conditions such as pH, concentration of the organic acids in the medium and salt composition in the medium will be identical for the fermentation and at the beginning of the electrolysis. The stream of fermentation broth after electrolysis may be advantageously recirculated to promote the dilution of the feed stream to the fermenter, in order to maintain the concentration of sugars or another substrate at the entry to the fermenter within a preferred range. In this manner, the feed stream containing, for example, from 10 to 30% by weight of sugars, is diluted until reaching a concentration of about 1 to 5% by weight. After the electrolysis, the fermentation broth may be transferred in the same conditions in which it was in the electrolysis reactor or may be cooled during the transfer.

If necessary, the fermentation broth stream leaving the electrochemical cell may be subjected to a process for the additional removal of the obtained products, for example by means of entrainment with inert gas, preferably carbon dioxide obtained as byproduct of the process, for the removal of residual contents of oxygen and recovery of the dissolved product. Subsequently, prior to being recirculated to the fermenter, the output stream from the electrolytic cell may be subjected to a step for the removal of eventual byproducts formed, such as, for example, alcohols, acetone, esters and alkanes, preferably by means of distillation. This stream may further undergo cooling with the use of a heat exchanger prior to being recirculated, particularly if the current density used in the electrolysis is high.

According to the present invention, the propionic acid produced is mainly converted to ethylene by means of the anodic decarboxylation reaction in an electrochemical cell located in the fermenter itself or in a separate reactor after the fermenter. The anodic decarboxylation reaction of organic acids in aqueous medium may result in the formation of various products, depending on the reaction conditions used, such as alkane dimers and olefins, or in the production of alcohols, in addition to hydrogen (H₂), carbon dioxide (CO₂), and a small amount of oxygen (O₂) and other hydrocarbons.

In the case of the anodic decarboxylation of propionic acid according to the process of the present invention, the main products obtained are ethylene, carbon dioxide and hydrogen, in addition to a small amount of byproducts, such as oxygen, ethanol, n-butane, ethane and ethyl propionate. In order to maximize the ethylene yield, the reaction of the present invention may use specific conditions. The conditions of the electrolysis step described herein minimize the formation of those undesired byproducts. The amount of hydrogen in relation to the propionic acid reacted along the course of the electrochemical formation of ethylene in the process according to the present invention is twice the amount when compared with the Kolbe reaction, aimed at the formation of n-butane.

The conditions of the electrolysis, either occurring within the fermenter or in a separate electrolytic reactor, should consist of at least a system comprised of two electrodes, designated as the anode and the cathode. Optionally, for the precise measurement and control of the potential of the cathode and the anode, a third electrode may be used, which is used as a reference and is selected from among the various ones heretofore described.

Various configurations of electrodes known in the art may be adequate to the present invention, such as, for example, fixed or movable electrodes, in shapes varying from flat to corrugated or tubular, in pairs or other configurations. In a preferred embodiment the electrodes, anode and cathode, are provided in pairs, forming a set of electrodes, such that the total electrical voltage of the reactor cell will correspond to the total sum of voltages between pairs of electrodes.

The anodes that are preferentially used are made of carbon or graphite. Alternatively, metallic anodes may be used, partially or entirely constituted of gold, platinum, nickel or other transition elements of the periodic table, or yet in the form of a conducting matrix deposited on a substrate, such as a matrix containing carbon, platinum, nickel or transition elements of the periodic table of elements. It is preferred that the anodes exhibit a rough finish that will provide a larger surface area.

The preferred cathodes are those that exhibit low overvoltage on release of hydrogen, such as those based on platinum, carbon, nickel, iron or alloys, such as duplex stainless steel. The electrode material may be solid or may be applied by coating on a supporting metal. The electrodes may optionally be kept apart by a separator in the form of a diaphragm, made of mineral and/or synthetic fibers, or synthetic membranes. The electrical current density should be between 0.1 and 1,000 mA/cm², preferably between 50 and 400 mA/cm² and the concentration of propionic acid in the medium should be between 1 and 80 g/L, preferably between 3 and 20 g/L. The anodic voltage should be between 1.0 and 5.0 Volts, preferably between 1.1 and 3.3 Volts.

The pH of the electrolysis should be between 4.0 and 7.5, preferably between 5.0 and 7.0, which facilitates the integration thereof with the fermentation process without requiring correction of the pH. The control of pH of the electrolysis is preferentially performed by removal of the propionic acid produced as a result of its anodic decarboxylation for the formation of ethylene. While the pH of the fermentation step lowers as a consequence of the formation of propionic acid, in the electrolysis the pH increases as a consequence of the consumption of the acid and its conversion to gaseous ethylene, thereby maintaining the stability of the integrated process within an optimal pH range, both for the fermentation and for the electrolysis. Optionally, a neutralizing base may also be added in order to maintain an optimal pH range.

Temperature does not have a significant influence on the electrolysis reaction; however, it implies a variation of viscosity and mass transfer. The range of operating temperatures of the electrolysis may vary from the temperature of the fermenter until 90 deg. C. Preferably, the temperature of the electrolysis should be between 20 and 45 deg. C. The pressure of this step also has little influence on the process. Preferably, the electrolysis is conducted at pressures near atmospheric pressure.

Since ethylene, which is the desired product resulting from the anodic decarboxylation reaction of propionic acid, is in gaseous state at the range of temperatures and pressures of the steps of the process according to the present invention, it thus exhibits low solubility in the medium. Therefore, the embodiments of the present invention allow the conduction of a fermentation process integrated with a step of anodic decarboxylation whereby the difficulties of recovery of the product are substantially reduced. Consequently, the cost of their removal thereof from the medium is minimized, and the inhibition of the fermentation by the drop in pH and/or by increase of concentration of the product is reduced. As a consequence of this low inhibition, the step of fermentation of the process according to the present invention affords high productivities of propionic acid, above 0.5 g/L.h, preferably above 1.0 g/L.h productivity.

The ethylene in gaseous form leaves the electrochemical cell together with carbon dioxide and hydrogen. The purification of the obtained ethylene may be achieved by means of separation processes known in the literature, such as distillation, extraction with solvents and/or adsorption of the impurities formed, by which ethylene with purity higher than 99.95% by weight may be obtained and it is thus adequate to the considerable purity demands of the polymerization processes.

Since the described process also provides hydrogen as a byproduct of the anodic decarboxylation reaction of the propionic acid, the hydrogen thus produced may be used, for example, in fuel cells for purposes of generation of electrical power for the process itself. Other applications for hydrogen are, for example, its use thereof as fuel for generation of steam and electrical power, or preferably, in the polymerization of ethylene and/or propylene to adjust the molecular weight of the polymer to be obtained.

Additionally, for the purpose of rendering the process according to the present invention self-sufficient in terms of energy, the hydrocarbons and the alcohols eventually produced as byproducts of the reaction may also be burned to generate energy, and the bagasse and the leaves resulting from the processing of the renewable natural raw material used as the sugar source may also be burned, thereby promoting an advantageous energy integration within the concept of the biorefinery. The electrical power obtained from co-generation with steam by means of the burning of residue (bagasse and leaves) and byproducts (hydrocarbons, alcohols and hydrogen) is sufficient to meet the requirements of the electrolysis reaction and of the existing equipment and machinery, thus rendering the integrated process self-sufficient in terms of energy.

In a similar manner, the butyric acid may be produced from the fermentation of natural carbon sources, such as sugars, glycerol, lactates or starch, in the presence of certain types of bacteria such as those of the genera Clostridium, Butyrivibrio and Butyribacterium, among others, such as, for example, the species Clostridium tyrobutyricum, Clostridium thermobutyricum, Clostridium butyricum, Clostridium aciditolerans, Clostridium kluyveri, Clostridium pasteurianum, Clostridium beijerinckii, Clostridium populeti, Clostridium acetobutylicum, Clostridium thermoamylolyticum, Fusobacterium nucleatum, Butyrivibrio fibrisolvens, Butyrivibrio methylotrophicum, Pseudobutyrivibrio ruminis, Eubacterium limosum, Thermoanaerobacter pseudethanolicus, Thermoanaerobacterium thermosulfurigenes, Clostridium thermocellum, and Clostridium thermoamylolyticum. Preferably, the fermentation to obtain butyric acid is conducted in the presence of bacteria selected from among the species Clostridium tyrobutyricum, Clostridium butyricum, Clostridium thermobutyricum, Clostridium aciditolerans, Clostridium acetobutylicum and Clostridium thermoamylolyticum. The fermentation typically occurs under anaerobic conditions.

Genetically modified microorganisms may be preferentially used for the purpose of reducing the formation of byproducts. One example of a genetically modified microorganism that is appropriate for the process according to the present invention is Clostridium tyrobutyricum ATCC 25755, an acidogenic bacterium that produces butyric acid, acetic acid, carbon dioxide and hydrogen. In this bacterium, the genes of the metabolic route of formation of acetic acid were deleted to maximize the production of butyric acid and hydrogen (Liu, X., et al, Enzyme and Microbial Technology, vol. 38, pp. 521-528, 2006).

Some of the important parameters in the fermentation according to the present invention are pH and temperature, and the optimal conditions for the fermentation of butyric acid are temperatures between 28 deg. C. and 45 deg. C., preferably between 30 deg. C. and 37 deg. C., and pH between 4.0 and 7.5, preferably between 5.0 and 7.0.

As far as it is know, a significant reduction of the pH value of the fermentation medium caused by the formation of the organic acids produces an inhibitory effect. Therefore, the fermentation of organic acids are normally conducted with rigid control of the pH value by means of the addition of bases, such as ammonium hydroxide, sodium hydroxide or calcium hydroxide, with the consequent formation of ammonium, sodium or calcium salts. In an embodiment of the present invention, the process may dispense the addition of bases for the control of the pH value of the medium, since the fermentation reaction is conducted with immediate removal of the carboxylic acids obtained from the fermentation medium by means of the anodic decarboxylation reaction, and thus eliminate the additional steps that are usually required for the recovery of the free organic acid. Optionally, and depending on the electro-decarboxylation conditions, a neutralizing base may also be added in order to maintain an optimal pH range. In this case, the additional steps required for the recovery of the organic acid are also eliminated, since the salt is also electro-decarboxylated to form the desired olefin.

A culture medium containing mineral salts and other nutrients is added to the fermentation system, and the stirring is maintained constant. The mineral salts and nutrients that are preferably used are, for example, sulfates, phosphates, nitrates, urea and ammonium, since these components do not influence the electrochemical step. Chlorides are to be avoided, since they interfere with the anodic decarboxylation reaction. When chlorides are necessary for the growth of the microorganism, an additional step for their removal may be added to the process, after the fermentation and before the electrochemical step. Chlorides may be removed by means of ion exchange resins, such as anionic or cationic resins or by processes such as electrodialysis or distillation.

The anaerobic condition of the system is maintained with a sprayer of nitrogen or, preferentially, the carbon dioxide gas obtained as a byproduct of the fermentation and/or of the electrodecarboxylation reaction.

According to the present invention, the butyric acid produced is converted mainly to propylene through the anodic decarboxylation reaction in an electrolysis reactor. The anodic decarboxylation reaction of organic acids in aqueous medium may result in the formation of various products, depending on the reaction conditions used, such as olefins and alkane dimers, or in the production of alcohols, in addition to hydrogen, carbon dioxide and a small amount of oxygen and other hydrocarbons. In the case of the anodic decarboxylation of butyric acid according to the process of the present invention, the main products obtained are propylene, hydrogen and carbon dioxide, in addition to a small amount of byproducts, such as n-hexane, oxygen, propanol, propane and cyclopropane.

A byproduct of the electrochemical reaction of butyric acid that may be present in the gaseous phase is cyclopropane, which may be easily isomerized to propylene. The isomerization reaction occurs at temperatures near 500 deg. C. and at pressures lower than atmospheric pressure. This reaction does not require any catalyst.

In order to maximize the propylene yield, the reaction according to the present invention should make use of specific conditions. The conditions of the electrolysis step described herein minimize the formation of those undesired byproducts. The amount of hydrogen in relation to the butyric acid reacted along the course of the electrochemical formation of propylene in the process according to the present invention is twice the amount when compared with the Kolbe reaction, aimed at the formation of n-hexane.

In a manner analogous to the anodic decarboxylation reaction of propionic acid, the anodic decarboxylation reaction of butyric acid may be conducted in the fermentation medium itself, and may occur either in the same section where the fermentation occurs or in a separate section, located contiguous to the fermentation region. Alternatively, the butyric acid may be electrodecarboxylated separately, at the end of batch fermentation, as shown in FIG. 3. Optionally, the fermentation broth remaining after the electrodecarboxylation reaction may be recirculated to the fermenter, thus promoting the dilution of the feed stream. Upon the electrolysis, the fermentation broth may be transferred in the same conditions found in the electrolyzer or it may be pre-cooled during the transfer.

In order to maximize the propylene yield, the anodic decarboxylation reaction of the butyric acid should make use of specific conditions. Different arrangements of electrodes known in the art may be adequate for the present invention, such as, for example, fixed or movable electrodes, or with flat, corrugated or tubular shapes, in pairs or other configurations. The conditions of the electrolysis, either occurring within the fermenter or in a separate electrolytic reactor, should preferably consist of at least a system comprised by two electrodes, designated as the anode and the cathode. Optionally, for a precise measurement and control of the potential of the anode and of the cathode, the use of a third electrode may be required, which is used as a reference and is selected from among the various ones heretofore described.

The anodes that are preferentially used are made of carbon or graphite. Alternatively, metallic anodes may be used, partially or entirely constituted of gold, platinum, nickel or other transition elements of the periodic table, or yet in the form of a conducting matrix deposited on a substrate, such as a matrix containing carbon, platinum, nickel or transition elements of the periodic table of elements. It is preferred that the anodes exhibit a rough finish that will provide a larger surface area.

The preferred cathodes are those that exhibit low overvoltage on release of hydrogen, such as those based on platinum, carbon, nickel, iron or alloys, such as duplex stainless steel. The electrode material may be solid or may be applied by coating on a supporting metal. The electrodes may optionally be kept apart by a separator in the form of a diaphragm, made of mineral and/or synthetic fibers, or synthetic membranes. The electrical current density should be between 0.1 and 1,000 mA/cm², preferably between 50 and 400 mA/cm² and the concentration of propionic acid in the medium should be between 1 and 80 g/L, preferably between 3 and 20 g/L. The anodic voltage should be between 1.0 and 5.0 Volts, preferably between 1.1 and 3.3 Volts.

The pH of the electrolysis should be between 4.0 and 7.5, preferably between 5.0 and 7.0, which facilitates the integration thereof with the fermentation process without requiring correction of the pH. The control of pH of the electrolysis is preferentially performed by removal of the butyric acid produced as a result of its anodic decarboxylation for the formation of propylene. While the pH of the fermentation step lowers as a consequence of the formation of butyric acid, in the electrolysis the pH increases as a consequence of the consumption of the acid and its conversion to gaseous propylene, thereby maintaining the stability of the integrated process within an optimal pH range, both for the fermentation and for the electrolysis. Optionally, a neutralizing base may also be added in order to maintain an optimal pH range.

The temperature does not have a significant influence on the electrolysis reaction; however, it implies a variation of viscosity and mass transfer. The range of operating temperatures of the electrolysis may vary from the temperature of the fermenter until 90 deg. C. Preferably, the temperature of the electrolysis should be between 20 and 45 deg. C. The pressure of this step also has little influence on the process. Preferably, the electrolysis is conducted at pressures near atmospheric pressure.

Since propylene, which is the desired product resulting from the anodic decarboxylation reaction of butyric acid, is in gaseous state at the range of temperatures and pressures of the steps of the process according to the present invention, it thus exhibits low solubility in the medium. Therefore, the embodiments of the present invention allow the conduction of a fermentation process integrated with a step of anodic decarboxylation whereby the difficulties of recovery of the product are substantially reduced. Consequently, the cost of their removal thereof from the medium is minimized, and the inhibition of the fermentation by the drop in pH and/or by increase of concentration of the product is reduced. As a consequence of this low inhibition, the step of fermentation of the process according to the present invention affords high productivities, above 2.0 g/L.h, preferably above 4.0 g/L.h of butyric acid productivity.

The propylene in gaseous form leaves the electrochemical cell together with carbon dioxide and hydrogen. The purification of the obtained propylene may be achieved by means of separation processes known from the literature, such as distillation, extraction with solvents and/or adsorption of the impurities formed, by which propylene with purity higher than 99.95% by weight may be obtained and it is thus adequate to the considerable purity demands of the polymerization processes.

In the case of the production of propylene, the described process provides hydrogen not only as a byproduct of the anodic decarboxylation reaction, but also as a co-product of the butyric fermentation. The hydrogen thus produced may be used, for example, in fuel cells for generation of electrical power to be used by the process itself Other applications for the hydrogen are, for example: the use as fuel in the generation of steam and electrical energy, or preferentially, in the polymerization of ethylene and/or propylene for adjustment of the molecular weight of the polymer to be obtained.

Additionally, for the purpose of rendering the process according to the present invention self-sufficient in terms of energy, the hydrocarbons and the alcohols eventually produced as byproducts of the reaction may also be burned to generate energy, and the bagasse and the leaves resulting from the processing of the renewable natural raw material used as the sugar source may also be burned, thereby promoting an advantageous energy integration within the concept of the biorefinery. The electrical power obtained from co-generation with steam by means of the burning of residue (bagasse and leaves) and byproducts (hydrocarbons, alcohols and hydrogen) is sufficient to meet the requirements of the electrolysis reaction and of the existing equipment and machinery, thus rendering the integrated process self-sufficient in terms of energy.

The fermentation for the production of organic acids according to the present invention may be conducted in a continuous process or a batch-feed process.

Eventually, the propionic and butyric acids produced in independent fermenters may be concurrently electrodecarboxylated in one single electrochemical cell.

Optionally, in association with the present invention, the carbonic gas produced either in the process of fermentation of carboxylic acids or in the electrochemical step, may be metabolized by microorganisms, such as algae, for the production of biofuels, or may be used in an entrainment column in order to remove the oxygen from the fermentation broth to be recirculated.

The ethylene and propylene produced by the process according to the present invention are 100% originated from renewable natural carbon sources, and may be advantageously used, among other uses, for the production of biopolymers and bio-copolymers of ethylene and/or propylene. As a consequence of this fact and of the integration thereof with byproducts of the processing of the renewable natural raw material and with byproducts of the present process, the production of the biopolymers by means of the use of the olefins obtained by the process according to the present invention promotes the absorption of carbon dioxide from the atmosphere during its life cycle, starting from the planting of the raw material. Additionally, the polymer of ethylene and/or propylene obtained by the method according to the present invention has the additional property of generating carbon dioxide of non-fossil origin upon its incineration.

The polyolefins produced from renewable raw materials, such as the biopolymers and the bio-copolymers of ethylene and/or of propylene obtained by the process according to the present invention may find use, preferably but without limitation, in the production of films, of fibers and of rigid objects produced by blow-molding, injection molding, thermoforming or rotational molding (rotomolding) techniques, particularly in the applications of:
diapers, tampons, sanitary and hygiene products,
carry bags, trash bags, industrial and agricultural films,
extrusion coatings,
non-woven webs,
rigid and flexible packages for foodstuffs, cosmetics, perfumes, cleaning products and sunscreen type protectors,
consumer goods in general such as jars, lids for vials, drinking glasses, bottles,
automotive parts such as air ducts, fuel tanks, water tanks, fenders and panels,
toys and games,
components or parts for electro-electronic equipment or electrical appliances,
pipes and fittings, flooring, linings, partitions, water tanks and cisterns,
boats and kayaks,
furniture,
synthetic grass fields and
carpets.

### EXAMPLE 1

Fermentation of Sugarcane Juice by Propionibacterium acidipropionici and Ethylene Production and Extraction by Anodic Electro-Decarboxylation Reaction

### Fermentation

A wild strain of Propionibacterium acidipropionici (ATCC No. 4875) was used to study propionic acid production using sugarcane juice as a carbon source. The bacterium was cultured in a medium containing sugar cane juice diluted in water and supplemented with 1 g/L of yeast extract. At dilution, the starting concentrations of sugars in the diluted sugarcane juice medium were measured at 38.5 g/L of sucrose, 9.9 g/L of glucose and 6. g/L of fructose. The medium was sterilized at 121° C. and 1 kgf/cm² for 20 min prior to use.

Free-cell batch fermentation was conducted in a 2.5 L bioreactor (Labfors—Infors HT) containing 1.0 L of the sterile medium inoculated with 100 ml. of pre-adapted cells of P. acidipropionici. The bioreactor temperature was maintained at 30 deg. C. and the agitation speed at 100 rpm. In this particular example, since the electrodecarboxylation reaction was performed in a separate reactor without broth recirculation and in order to maximize propionic acid yield, a constant pH of 6.5 was automatically controlled by adding a 4M NaOH solution. Anaerobic conditions were maintained through the use of a N₂ atmosphere.

Batch fermentation was stopped after 132 h and the products in the liquid were quantified through High Performance Liquid Chromatography (HPLC) coupled to a Refraction Index detector and using standards for the desired metabolites (Alliance HT—Waters Chromatographer using a Aminex HPX-87H Organic Acid Column from BioRad, operating at 35 deg. C. and using 4 mM H₂SO₄ as the eluent at a flux of 0.6 mL/min). Table 1 shows the final concentration of the products.

TABLE 1
Final product concentrations after 132 h of fermentation by
Propionibacterium acidipropionici (ATCC No. 4875) of
sugarcane juice media (see composition in text) under
controlled conditions of temperature, pH and agitation.

| Component | Concentration (g/L) |
|---|---|
| Propionic acid | 21.3 |
| Acetic acid | 7.9 |
| Succinic acid | 3.7 |

### Anodic Electro-Decarboxylation Reaction

The centrifuged and filtered fermentation broth (250 ml) was subjected to the anodic electro-decarboxylation reaction. The electrochemical cell consists of a cylindrical glass vessel containing two parallel electrodes, placed 4 mm apart. The anode was a graphite bar (21.6 cm²) and the cathode was a stainless steel bar. Electrolysis was conducted at 300 rpm for 6 hours at a current density of 10 mA/cm² and 25 deg. C., using an Autolab PGSTAT 302N potentiostat. Prior to the electrolysis, the fermentation broth was purged with nitrogen gas for 15 min in order to obtain an inert atmosphere.

Gas chromatography analyses were performed on an Agilent 7890A apparatus equipped with a split/splitless injector, FID and TCD detectors. Injector and detectors temperatures were 150 and 300 deg. C., respectively. The capillary column was an Agilent 19095P-Q04 (30 m×530 μm×40 μm), the flow rate of carrier gas (helium) was 4.1 mL/min, and the oven temperature was programmed from 35 deg. C. (3 min) to 60 deg. C. (9 min) at 40 deg. C./min, and then to 230 deg. C. (4 min) at 40 deg. C./min. Chromatographic data acquisition and processing were carried out using GC ChemStation software revision B.04.02 (Agilent).

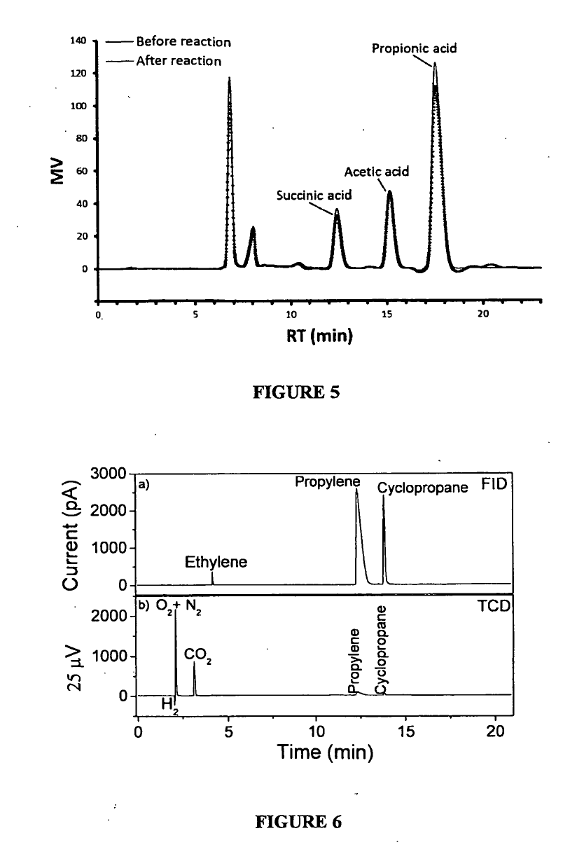

During the experiment, the gas was analyzed in steps of 30 minutes through automatic injection into the GC system. FIG. 4 shows the gas analysis detected by (a) FID and (b) TCD after 6 hours of experiment. Only the expected peaks (ethylene, H₂ and CO₂ from anodic decarboxylation reaction, and N₂, from purge) were detected in all chromatograms obtained during the experiment. The electrochemical reaction was stopped after 6 h and the products in the liquid phase were quantified through HPLC and the final pH was measured. FIG. 5 shows the HPLC chromatogram of the fermentation broth before (solid line) and after the anodic electro-decarboxylation reaction (dotted line). The analysis of the HPLC chromatograms showed that 2.25 g/l of propionic acid was consumed during the reaction and the pH increased from 6.5 to 7.5. The absence of new peaks in the liquid phase after the reaction demonstrates that the consumed propionic acid was totally converted to compounds in gaseous form. The raise in pH is consistent with the removal of the carboxylic acids by the anodic decarboxylation reaction.

### EXAMPLE 2

### Propylene Production and Extraction by Anodic Electro-Decarboxylation Reaction of Butyric Acid

A solution of 250 mL containing 10.5 g/L of butyric acid and with pH adjusted to 5 with the addition of 10 M KOH was subjected to the anodic electro-decarboxylation reaction. The electrochemical cell consists of a cylindrical glass vessel containing two parallel electrodes, placed 4 mm apart. The anode was a graphite bar (21.6 cm²) and the cathode was a stainless steel bar. Electrolysis was conducted at 300 rpm for 6 hours at a current density of 10 mA/cm² and 25 deg. C., using an Autolab PGSTAT 302N potentiostat. Prior to the electrolysis, the solution was purged with nitrogen gas for 15 min in order to obtain an inert atmosphere.

Gas chromatography analyses were performed on an Agilent 7890A apparatus equipped with a split/splitless injector, FID and TCD detectors. Injector and detectors temperatures were 150 and 300 deg. C., respectively. The capillary column was an Agilent 19095P-Q04 (30 m×530 μm×40 μm), the flow rate of carrier gas (helium) was 4.1 mL/min, and the oven temperature was programmed from 35 deg. C. (3 min) to 60 deg. C. (9 min) at 40 deg. C./min, and then to 230 deg. C. (4 min) at 40 deg. C./min Chromatographic data acquisition and processing were carried out using GC ChemStation software revision B.04.02 (Agilent).

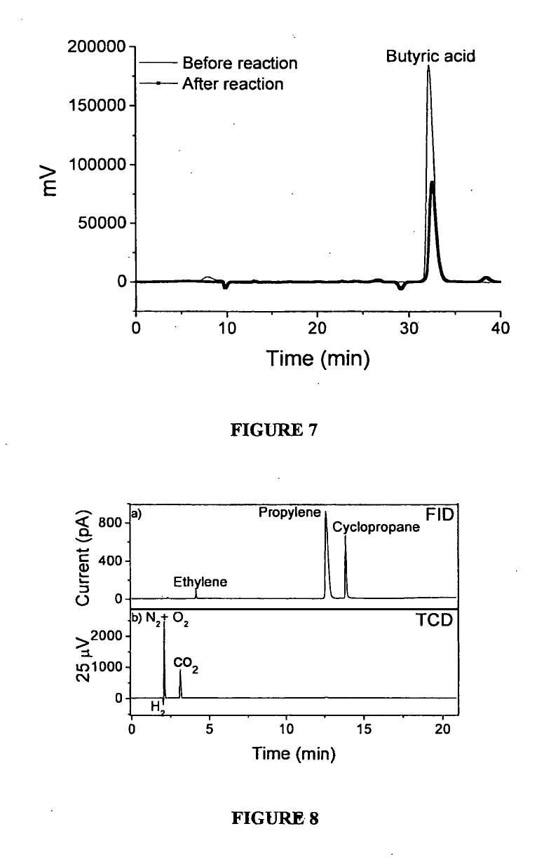

During the experiment, the gas was analyzed in steps of 30 minutes through automatic injection into the GC system. FIG. 6 shows the gas analysis detected by (a) FID and (b) TCD after 6 hours of experiment. The expected peaks (propylene, H₂ and CO₂ from anodic decarboxylation reaction, and N₂, from purge) and small peak of ethylene were detected in all chromatograms obtained during the experiment. The electrochemical reaction was stopped after 6 h, the final pH was measured and the products in the liquid phase were quantified through High Performance Liquid Chromatography coupled to a Refraction Index detector and using standards for the desired metabolites (Varian Chromatographer using a Aminex HPX-87H Organic Acid Column from Transgenomic, operating at room temperature and using 0.002 M H₂SO₄ as the eluent at a flux of 0.6 mL/min) FIG. 7 shows the HPLC chromatogram of the solution before (solid line) and after the anodic electro-decarboxylation reaction (dotted line). The analysis of the HPLC chromatograms showed that 6.1 g/L of butyric acid was consumed during the reaction and the pH increased from 5.06 to 7.5. One new peak was observed in the liquid phase after the reaction at a retention time of 39 min. and may correspond to propanol. Propanol may be formed through the electrodecarboxylation reaction once the pH raises above 7. Nevertheless, the proportion of this new compound, when compared to the main products propylene and cyclopropane, is negligible under the conditions used. The raise in pH is consistent with the removal of the carboxylic acids by the anodic decarboxylation reaction.

### EXAMPLE 3

Fermentation of Sugarcane Juice by Clostridium butyricum and Prophylene Production and Extraction by Anodic Electro-Decarboxylation Reaction

### Fermentation

A wild strain of Clostridium butyricum (DSM No. 10702) was used to study butyric acid production using sugarcane juice as a carbon source. The bacterium was cultured in a medium containing sugar cane juice diluted in water and supplemented with 0.5 g/L yeast extract, 0.5 g/L peptone, 1.0 g/L trypticase, 0.5 g/L KCl and 500 mg/L cysteine hydrochloride. At this dilution, the starting concentrations of sugars in diluted sugarcane juice medium were measured at 24.9 g/L of sucrose, 3.6 g/L of glucose and 1.9 g/L of fructose. The medium was sterilized at 121deg. C. and 1 kgf/cm² for 20 min prior to use.

Free-cell batch fermentation was conducted in a 2.5 L bioreactor (Labfors—Infors HT) containing 1.0 L of the sterile medium inoculated 100 ml of adapted cells of C. butyricum. The bioreactor temperature was maintained at 37 deg. C. and the agitation speed at 150 rpm. The initial pH was 7.0 and after decrease to 5.0 was kept in this value with the addition of 4 M KOH. Anaerobic conditions were maintained through the use of a N₂ atmosphere.

Batch fermentation was stopped after 117 h and the products were quantified through High Performance Liquid Chromatography (HPLC) coupled to a Refraction Index detector and using standards for the desired metabolites (AllianceHT—Waters Chromatographer using a Aminex HPX-87H Organic Acid Column from Bio-Rad, operating at 35 deg. C. and using 4 mM H₂SO₄ as the eluent at a flux of 0.6 mL/min). Table 2 shows the final concentration of the products.

TABLE 2
Final product concentrations after 117 h of fermentation by
Clostridium butyricum (DSM No. 10702) of sugarcane
juice media (see composition in text) under controlled
conditions above described

| Component | Concentration (g/L) |
|---|---|
| Butyric acid | 6.0 |
| Acetic acid | 3.7 |

### Anodic Electro-Decarboxylation Reaction

The centrifuged and filtered fermentation broth (250 ml) was distilled at 100 deg. C. and subjected to the anodic electro-decarboxylation reaction. The electrochemical cell consists of a cylindrical glass vessel containing two parallel electrodes, placed 4 mm apart. The anode was a graphite bar (21.6 cm²) and the cathode was a stainless steel bar. Electrolysis was conducted at 300 rpm for 6 hours at a current density of 10 mA/cm² and 25 deg. C., using an Autolab PGSTAT 302N potentiostat. Prior to the electrolysis, the distilled fermentation broth was purged with nitrogen gas for 15 min in order to obtain an inert atmosphere.

Gas chromatography analyses were performed on an Agilent 7890A apparatus equipped with a split/splitless injector, FID and TCD detectors. Injector and detectors temperatures were 150 and 300 deg. C., respectively. The capillary column was an Agilent 19095P-Q04 (30 m×530 μm×40 μm), the flow rate of carrier gas (helium) was 4.1 mL/min, and the oven temperature was programmed from 35° C. (3 min) to 60 deg. C. (9 min) at 40 eg. C./min, and then to 230 deg. C. (4 min) at 40 deg. C./min. Chromatographic data acquisition and processing were carried out using GC ChemStation software revision B.04.02 (Agilent).

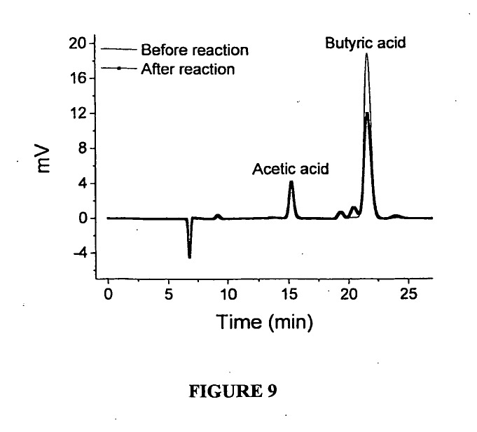

During the experiment, the gas was analyzed in steps of 30 minutes through automatic injection into the GC system. FIG. 8 shows the gas analysis detected by (a) FID and (b) TCD after 6 hours of experiment. The expected peaks (propylene, H₂ and CO₂ from anodic decarboxylation reaction, and N₂, from purge) and a small peak of ethylene were detected in all chromatograms obtained during the experiment. The electrochemical reaction was stopped after 6 h and the products in the liquid phase were quantified through HPLC and the final pH was measured. FIG. 9 shows the HPLC chromatogram of the fermentation broth before (solid line) and after the anodic electro-decarboxylation reaction (dotted line). The analysis of the HPLC chromatograms showed that 1.2 g/L of butyric acid was consumed during the reaction and the pH increased from 4.5 to 6.0. One new peak was observed in the liquid phase after the reaction at a retention time of 39 min that may correspond to propanol. Propanol may be formed through the electrodecarboxylation reaction once the pH raises above 7. Nevertheless, the proportion of this new compound formed, when compared to the main products propylene and cyclopropane, is negligible under the conditions used. The raise in pH is consistent with the removal of the carboxylic acids by the anodic decarboxylation reaction.

1. A method for the production of olefins, comprising the production of carboxylic acids from the fermentation of at least one organic substrate from a renewable and natural raw material, followed by the subsequent anodic decarboxylation of the carboxylic acids thus obtained for forming the olefins.

2. The method of claim 1, wherein the fermentation is conducted with immediate removal from the fermentation medium of the carboxylic acids thus obtained by means of the anodic decarboxylation reaction.

3. The method of claim 1 or 2, wherein the control of the pH in the fermentation medium takes place by means of the production of olefins generated by the anodic decarboxylation of the organic acids thereof.

4. The method of claim 1 or 2, wherein the control of the pH in the fermentation medium takes place by a combination of the anodic decarboxylation of the organic acids thereof and by the addition of a neutralizing base.

5. The method of claim 1 or 2, wherein the carboxylic acid obtained by fermentation is the propionic acid, which generates ethylene through the anodic decarboxylation thereof.

6. The method of claim 1 or 2, wherein the carboxylic acid obtained by fermentation is the butyric acid, which generates propylene through the anodic decarboxylation thereof.

7. The method of claim 1 or 2, wherein the olefins are butylene or pentene, which are obtained by means of anodic decarboxylation, respectively from valeric or caproic acids.

8. The method of claim 1, wherein the organic substrates from renewable and natural raw material are selected from among starch, cellulose, hemicellulose, glycerol, sorbitol, lactose, lactates and sugars.

9. The method of claim 8, wherein the organic substrates from renewable and natural raw material are sugars.

10. The method of claim 5, wherein the propionic acid is obtained by fermentation in the presence of bacteria of the genus Propionibacterium.

11. The method of claim 10, wherein the bacterium is Propionibacterium acidipropionici.

12. The method of any one of claims 5, 10 or 11, wherein the fermentation of propionic acid occurs at temperatures between 28 deg. C. and 45 deg. C. and at a pH between 4.0 and 7.5.

13. The method of claim 12, wherein the fermentation of propionic acid occurs at temperatures between 30 deg. C. and 37 deg. C. and at a pH between 5.0 e 7.0.

14. The method of claim 6, wherein the butyric acid is obtained by fermentation, in the presence of bacteria such as those of the genera Clostridium, Butyrivibrio and Butyribacterium.

15. The method of claim 14, wherein the bacterium is Clostridium tyrobutyricum, Clostridium butyricum, Clostridium thermobutyricum, Clostridium aciditolerans, Clostridium acetobutylicum or Clostridium thermoamylolyticum.

16. The method of any one of claims 6, 14 or 15, wherein the fermentation of butyric acid occurs at temperatures between 28 deg. C. and 45 deg. C. and at a pH between 4.0 and 7.5.

17. The method of claim 16, wherein the fermentation of butyric acid occurs at temperatures between 30 deg. C. and 37 deg. C. and at a pH between 5.0 and 7.0.

18. The method of claim 1 or 2, wherein the anodic electrodecarboxylation of the carboxylic acids may be conducted in the fermenter itself, and may take place either in the same region wherein the fermentation occurs or in a separate section, contiguous to the fermentation region, or in a separate electrolysis reactor.

19. The method of any one of claim 1, 2 or 18, wherein the fermentation broth stream, upon the anodic electrodecarboxylation thereof, may be recirculated to the feed stream of the fermenter.

20. The method of claim 19, wherein the stream to be recirculated is cooled with the use of a heat exchanger prior the recirculation to the feed stream of the fermenter.

21. The method of claim 19, wherein the stream to be recirculated to the feed is distilled for removal therefrom of any byproducts eventually formed.

22. The method of any one of claims 1 to 21, wherein the anodic electro-decarboxylation comprises an anode and a cathode, an electrical current density between 0.1 and 1,000 mA/cm², an anodic voltage between 1.0 and 5.0 Volts and a pH between 4.0 and 7.5.

23. The method of claim 22, wherein the anode is made of carbon or graphite, the cathode evidences low overvoltage on release of hydrogen, the electrical current density is between 50 and 400 mA/cm², the anodic voltage is between 1.1 and 3.3 Volts and the pH is between 5.0 and 7.0.

24. The method of any one of claims 1 to 23, wherein the operating temperature of the anodic decarboxylation may vary from the temperature of the fermenter until 90 deg. C.

25. The method of claim 5, wherein the concentration of propionic acid in the medium for the anodic electrodecarboxylation is between 1 and 80 g/L.

26. The method of claim 25, wherein the concentration of propionic acid in the medium is between 3 and 20 g/L.

27. The method of claim 6, wherein the concentration of butyric acid in the medium for the anodic electrodecarboxylation is between 1 and 40 g/L.

28. The method of claim 27, wherein the concentration of butyric acid in the medium is between 3 and 20 g/L.

29. An olefin, comprising being produced from at least one raw material from a renewable and natural raw material by means of the method as defined in any one of claims 1 to 26.

30. The olefin of claim 29, wherein it consists of ethylene or propylene.

31. A polyolefin, comprising being generated from an olefin as defined in claim 29 or in claim 30, and having the ability to generate carbon dioxide of non-fossil origin upon incineration thereof.

32. The polyolefin of claim 31, wherein it consists of polyethylene, polypropylene or copolymers thereof.

33. A use of the polyolefin of claim 31 or 32, wherein occurring in the production of films, fibers and rigid objects produced by blow molding, injection molding or rotational molding (rotomolding) techniques.

34. The use of the polyolefin of claim 33, wherein occurring in the applications of:
diapers, tampons, sanitary and hygiene products,
carry bags, trash bags, industrial and agricultural films,
extrusion coatings,
non-woven webs,
rigid and flexible packages for foodstuffs, cosmetics, perfumes, cleaning products and sunscreen type protectors,
consumer goods in general such as jars, lids for vials, drinking glasses, bottles,
automotive parts such as air ducts, fuel tanks, water tanks, fenders and panels,
toys and games,
components or parts for electro-electronic equipment or electrical appliances,
pipes and fittings, flooring, linings, partitions, water tanks and cisterns,
boats and kayaks,
furniture,
synthetic grass fields and
carpets.
